(19) United States
(12) Patent Application Publication
(10) Pub. No.: US 2024/0174718 A1
(43) Pub. Date: May 30, 2024

(54) RYANODINE RECEPTOR INHIBITOR COMPOUNDS AND METHODS RELATING THERETO

(71) Applicant: Vanderbilt University, Nashville, TN (US)

(72) Inventors: Jeffrey N. Johnston, Nashville, TN (US); Suzanne Batiste, Nashville, TN (US); Abigail N. Smith, Nashville, TN (US); Daniel J. Blackwell, Nashville, TN (US); Björn C. Knollmann, Bethesda, MD (US)

(21) Appl. No.: 18/517,872

(22) Filed: Nov. 22, 2023

Related U.S. Application Data
(60) Provisional application No. 63/427,123, filed on Nov. 22, 2022.

(51) Int. Cl. C07K 11/02; A61K 45/06; A61P 9/06

(52) U.S. Cl. CPC C07K 11/02; A61K 45/06; A61P 9/06

(57) ABSTRACT

Anodine receptor type 2 (RyR2) inhibitors and methods of their use in the treatment of cardiac conditions.

### PRIOR APPLICATIONS

This application claims benefit to U.S. Patent Application No. 63/427,123, filed Nov. 22, 2022, the contents of which are incorporated herein by reference.

### FIELD OF THE INVENTION

The present invention relates to the field of novel anodine receptor type 2 (RyR2) inhibitors and methods of their use in the treatment of cardiac conditions. In general, the RyR2 inhibitors of the present invention assist in the normalization of intracellular calcium homeostasis. In embodiments of the present invention, compounds of the present invention inhibit mammalian cardiac ryanodine receptor calcium release channels

### BACKGROUND AND SUMMARY OF THE INVENTION

Heart failure is a common, progressive and often fatal cardiac condition. Heart failure is responsible for millions of hospitalizations and over 300,000 deaths in the U.S. and over a million in Western countries annually, resulting in an enormous impact on public health. A leading cause of death in patients with heart failure is arrhythmia. A primary therapeutic strategy over the last several decades has been to suppress arrhythmias by the use of anti-arrhythmic drugs. There are currently several types of drugs available on the market to treat various types of arrhythmias.

Of a number of anti-arrhythmic drugs used over the past several decades, β-blockers are a class of anti-arrhythmic drugs proven to have significant survival benefits, and have become the most widely used drugs for the treatment of heart failure and cardiac arrhythmias. Although β-blockers have revolutionized the treatment of patients with these diseases, the absolute benefit of β-blocker treatment is low (^{˜}7% absolute reduction in all-cause mortality). Clearly, there is a long felt need in the art for new anti-arrhythmic drugs with better efficacy.

Hit-to-lead studies employ a variety of strategies to optimize binding to a target of interest. When a structure for the target is available, hypothesis-driven structure-activity relationships (SAR) are a powerful strategy for refining the pharmacophore to achieve robust binding and selectivity characteristics necessary to identify a lead compound. Recrafting the three-dimensional space occupied by a small molecule, optimization of hydrogen bond contacts, and enhancing local attractive interactions are traditional approaches in medicinal chemistry. Ring size, however, is rarely able to be leveraged as an independent variable since most hits lack the symmetry required for such a study. The discovery that the cyclic oligomeric depsipeptide ent-verticilide inhibits mammalian cardiac ryanodine receptor calcium release channels with sub-micromolar potency provides an opportunity to explore ring-size as a variable, independent of other structural or functional group changes. The present inventors discovered the importance of ring size as an independent variable, suggesting that modest conformational changes alone can dramatically affect potency.

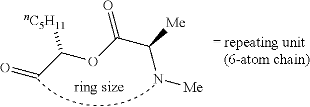

One embodiment of the present invention is a compound of the following formula:

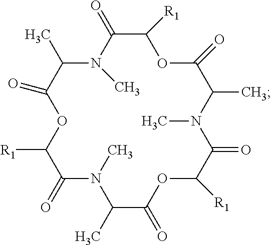

wherein each R₁ is independent and chosen from C₁-C₁₂ substituted or unsubstituted alkyl; and pharmaceutically acceptable salts thereof.

### BRIEF DESCRIPTION OF THE DRAWINGS

FIG. 1 shows structures of compounds studied by the inventors, including compounds of the present invention.

FIG. 2 shows an exemplary scheme for preparing compounds of the present invention.

FIGS. 3(A)-3(E) show a biological screen for cardiac ryanodine receptor (RyR2) activity. Note that ‘ent-24’=9, and ‘ent-18’ refers to structure 8 in FIG. 1, which is also the enantiomer of a natural product. (3A) Calcium spark frequency in permeabilized murine cardiomyocytes was recorded as an index of RyR2 activity. Compounds were screened at 25 uM concentration after 10-minute incubation. *=p<0.001 by one-way ANOVA with Tukey's post-hoc test. (3B) Incubation time was extended to 60 minutes for the 12- and 30-membered rings. (3C) The seco-acid precursors to 12- and 18-membered rings were tested at 25 uM. (3D) Concentration response curve for the 18-membered ring and (3E) 24-membered ring. Cells were incubated for 30 minutes

FIGS. 4(A-G) show intracellular Ca2+ measurements from intact Casq2−/− cardiomyocytes. FIG. 4(A) shows diastolic Ca2+ measured during field stimulation. FIG. 4(B) shows paced Ca2+ transient amplitudes. FIG. 4(C) shows time to peak and (D) Ca2+ decay kinetics of paced Ca2+ transients. FIG. 4(E) shows caffeine-induced Ca2+ transient amplitude. FIG. 4(F) shows caffeine-induced Ca2+ transient decay kinetics. FIG. 4(G) shows fractional Ca2+ release (paced transient amplitude/SR Ca2+ content). For panels A-D and G, N=26, 30, 30, 30, and 31 cells for DMSO, 0.03, 0.1, 1, and 3 μM ent-B1, respectively. For panels E and F, N=14, 27, 29, 29, and 30 cells for DMSO, 0.03, 0.1, 1, and 3 μM ent-B1, respectively. Data are shown as mean±SD with individual data points.

FIGS. 5(A-E) show action potential measurements in intact Casq2^{-/-} ventricular myocytes. FIG. 5(A-B) Representative membrane potential recordings from current-clamped myocytes stimulated at 1 Hz. Myocytes were pre-incubated for 2½ hrs with vehicle (DMSO) (A) or 0.3 μM ent-B1 (B) before patch clamping individual myocytes. Dotted line indicates 0 mV. FIG. 5(C-E) Summary of action potential durations (APD_{30, 50 & 90}). N=14 cells from 2 Casq2^{-/-} mice each. Individual data points are shown with mean±SD. All data were analyzed using unpaired t test.

FIG. 6(A-C) show the effect of ent-B1 on heart rate of Casq2^{-/-} mice. Mice were given vehicle (DMSO) or ent-B1 by intraperitoneal injection 15 min before isoproterenol challenge. FIG. 6(A) shows baseline heart rate (beats per minute). FIG. 6(B) shows peak heart rate and FIG. 6(C) shows change in heart rate following intraperitoneal administration of 3 mg/kg isoproterenol. Mixed effects model pre-test P>0.05 for period, sequence, and treatment in all groups in each panel.

FIG. 7 shows the effect of ent-B1 on electrocardiogram (ECG) parameters of Casq2^{-/-} mice. Mice were given vehicle (DMSO) or ent-B1 by intraperitoneal injection 15 min before isoproterenol challenge. FIG. 7(A) PR, FIG. 7(B) QRS, and FIG. 7(C) show QT intervals were measured during baseline ECG recording 2 minutes prior to administration of 3 mg/kg isoproterenol. Mixed effects model pre-test P>0.05 for period, sequence, and treatment in all groups in each panel.

FIGS. 8(A-D) show RyR2 single channel recording in artificial lipid bilayers. 7(A) shows the chemical structure of 24-membered cyclic depsipeptide ent-1 and an embodiment of the present invention, an 18-membered cyclic depsipeptide ent-B1. 8(B) shows a representative trace of RyR2 single channel recording before and after application of ent-B1 at +40 mV, with 2 mM ATP and 100 nM Ca (cis) and 1 mM Ca (trans). Channel openings are in the upward direction (c, closed; o, open). 8(C) shows open probability of RyR2 in the presence of ent-B1 relative to vehicle (DMSO). Fitting values using non-linear regression to a Hill-function (line) yielded an IC₅₀ of 0.24 μM [95% CI: 0.08-1.00] and a maximal inhibitory effect Iₘₐₓ of 61% (95% CI: 52%-72%) 8(D) Probability distributions of current amplitude of 30-second segments of recording from (B) before and after addition of 30 μM ent-B1.

FIG. 9 shows the effect of an example of the present invention on ent-B1 on RyR2 activity in porcine SR vesicles. RyR2 activity was quantified by the fraction of [³H]ryanodine bound to RyR2. The concentration-response curve shows results for ent-B1 relative to vehicle (DMSO). ent-B1 IC₅₀=1.93 μM (95% CI 0.74-4.62 μM), Iₘₐₓ=29%. Data are shown as mean±SD (n=6).

FIGS. 10(A-B) show the spontaneous Ca²⁺ release in intact mouse Casq2^{-/-} cardiomyocytes. 10(A) shows the representative fluorescence recordings from ventricular myocytes isolated from Casq2^{-/-} mice. Cells were field stimulated at 3 Hz for 20 s before recording. Blue marks indicate final stimulations of pacing train, arrows SCR events. Recording duration was 40 s before 10 mM caffeine application to assess total SR Ca²⁺ content. 10(B) shows SCR frequency concentration-response curve for ent-B1 following 20 s pacing protocol. ent-B1 data normalized to vehicle (DMSO). Data are shown as mean±SD. Fitting values using non-linear regression to a Hill-function yielded an IC₅₀ of 0.23 μM [95% CI: 0.099-0.63] and a maximal inhibitory effect Iₘₐₓ of 62% (95% CI: 53%-89%) N=26, 30, 30, 30, and 31 cells for 0, 0.03, 0.1, 0.3, and 1 μM ent-B1 respectively.

FIGS. 11(A-B) show in vivo pharmacokinetics in mice for a compound of the present invention. FIG. 11(A) shows plasma concentrations of ent-B1 after intraperitoneal administration of 3 mg/kg dose. N=3 mice. Plasma collected serially at 0.167, 0.333. 0.5, 1, 3, and 8 hours post-administration. 11(B) shows quantification of ent-B1 incubated ex vivo in murine plasma for 2 hours at 37 deg C. Individual data points (grey) shown alongside mean values with connecting lines.

FIGS. 12(A-D) show antiarrhythmic efficacy for a compound of the present invention in CPVT mice. Triple crossover study design for in vivo arrhythmia challenge. N=22 Casq2^{-/-} mice were randomly assigned to vehicle (DMSO), 3 mg/kg, or 30 mg/kg ent-B1 and crossed over twice with one-week washouts between treatment. Mice underwent catecholamine-induced arrhythmia challenge with isoproterenol 15 min after intraperitoneal drug administration. 12(A) shows sample ECG traces of normal rhythm (top) and arrhythmias including PVCs and couplets (middle), and bigeminy and ventricular tachycardia (bottom). Ectopic beats are denoted by p (PVC), c (couplet), and vt (ventricular tachycardia). Normal sinus beats are not marked. 12(B) shows total number of ventricular ectopic beats per mouse. Individual data are shown with median and interquartile range. Bonferroni-adjusted P values by pairwise Wilcoxon matched-pairs signed rank test. *P=0.020 vs Veh; #P=0.039 vs Veh. 12(C) shows incidence of ventricular tachycardia. #P=0.034 vs Vehicle by Fisher's exact test. 12(D) shows arrhythmia risk scores were based on an ordinal scale of: 4=ventricular tachycardia, 3=couplet, 2=bigeminy, 1=isolated PVC, 0=no PVCs. Bonferroni-adjusted P values by pairwise Wilcoxon matched- pairs signed rank test. #P=0.0058 vs Vehicle.

### DESCRIPTION OF THE INVENTION:

Patients with heart rhythm disorders have a major, unmet need for new treatments. Among all causes of mortality in the United States, sudden cardiac death (SCD)—a result of ventricular arrhythmias—is responsible for 15% of all deaths annually. With the exception of beta-adrenergic receptor inhibitors, none of the agents marketed as antiarrhythmic drugs in the U.S. prevent SCD, with most of them increasing rates of mortality in patients with ischemic heart disease or heart failure. The common mechanism of action shared by FDA-approved antiarrhythmic drugs is the modulation of ion channels or G-protein coupled receptors expressed in the cell membrane. As such, better antiarrhythmic drugs are needed. The present invention helps meet that need.

RyR2s are Ca²⁺ release channels located in the membrane of the sarcoplasmic reticulum. The mechanism of Ca²⁺-induced Ca²⁺ release to facilitate excitation-contraction (EC) coupling in cardiomyocytes is well-studied, with RyR2 serving as the release channel for SR Ca²⁺ stores. Pathologic Ca²⁺ release from RyR2 has been reported in both genetic and acquired arrhythmia disorders through gain of function mutations or post-translational modifications to RyR2, respectively. In either scenario, an increased open probability of RyR2 causes Ca²⁺ to “leak” from the SR, which disrupts both the temporal and functional integrity of cardiac Ca²⁺ handling. Specifically, increased [Ca²⁺]_{cytosolic} is pumped extracellularly through the electrogenic Na⁺/Ca²⁺ exchanger, leading to delayed afterdepolarizations driven by the untimely influx of Na⁺ as Ca²⁺ homeostasis is restored.

As stated above, an embodiment of the present invention is a compound of the following formula:

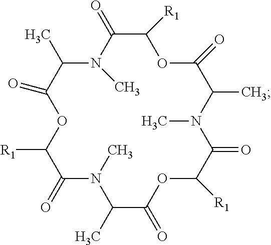

wherein each R₁ is independent and chosen from C₁-C₁₂ substituted or unsubstituted alkyl; and pharmaceutically acceptable salts thereof.

In another embodiment, R₁ is C₁-C₆ alkyl. In another, R₁ is C₁-C₅ alkyl. In another, R₁ is C₅H₁₁. Of course, one of ordinary skill in the art would understand that in all instances described herein, while each R₁ may be the same, the R₁ variables are always independent.

In another embodiment, the present invention relates to a method of treating a subject suffering from a cardiac condition associated with RyR2, comprising administering to a subject in need thereof a RyR2 inhibiting effective amount of a compound of the present invention.

In one aspect, the cardiac condition is heart failure or arrhythmia.

Another embodiment of the present invention is a method of treating a subject suffering from a cardiac condition associated with RyR2, comprising the step of co-administering to a subject in need thereof a RyR2 inhibiting effective amount of a compound of the invention with a drug having a known effect of treating a cardiac condition. The cardiac condition may be heart failure or arrhythmia.

In some embodiments, the drug may be an anti-arrhythmia agent. In others, the drug is a beta-blocker.

As used herein, “ryanodine receptor type 2 inhibitor” or “RyR2 inhibitor” refers to a compound that modulates intracellular calcium release, such as calcium release modulated by the sarcoplasmic reticulum (SR), in cardiac muscle. A RyR2 inhibitor may, for example, modulate calcium release to achieve or maintain intracellular calcium homeostasis in cardiac muscle. A RyR2 inhibitor may suppress store-overload-induced Ca²⁺ release (SOICR). A RyR2 inhibitor may suppress SOICR while minimally inhibiting or not inhibiting Ca²⁺-induced Ca²⁺ release (CICR), as described herein.

As used herein, “minimal inhibition of CICR activity” and variants thereof refers to an extent of inhibition that does not considerably alter the excitation-contraction coupling or the normal function of cardiac cells. Minimal inhibition of CICR activity may refer to, in certain embodiments, about or at most about 5%, 10%, 15%, 20%, 25%, 30%, 35%, or 40% inhibition, or any range derivable therein, of CICR activity.

As used herein, “store-overload-induced Ca²⁺ release” or “SOICR” refers to the spontaneous release of Ca²⁺ by the sarcoplasmic reticulum (SR) under the conditions of Ca²⁺ overload as a result of activation of the ryanodine receptor type 2 (RyR2) channel by SR luminal Ca²⁺.

As used herein, “RyR2” refers to the ryanodine receptor type 2 protein, wild-type or mutant, found primarily in heart tissue, but other tissues as well.

As used herein, “a condition associated with RyR2” refers to a disorder or disease that can be treated and/or prevented by modulating RyR2 (ryanodine receptor type 2) that regulates calcium homeostasis in cells. Conditions associated with RyR2 include, for example, cardiac disorders and diseases, which are described in more detail herein.

As used herein, “conditions” comprise diseases, disorders, syndromes, etc. as applicable.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active.

Compounds of the present invention may contain one or more asymmetric centers and thus can occur as racemates and racemic mixtures, single enantiomers, diastereomeric mixtures and individual diastereomers. All possible stereoisomers of the compounds of the present invention are contemplated as being within the scope of the present invention. The chiral centers of compounds of the present invention can have the S- or the R-configuration, as defined by the IUPAC 1974 Recommendations. The present invention is meant to comprehend all such isomeric forms of the compounds of the invention.

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, tartrates, citrates, hydrohalides, phosphates and the like. The term “pharmaceutically acceptable salts” refers to salts prepared from pharmaceutically acceptable non-toxic bases or acids including inorganic or organic bases and inorganic or organic acids, and are described in more detail herein.

As used herein, “alkyl” refers to a saturated or unsaturated, straight- or branched-chain radical containing from 1 to 30 carbon atoms. Examples of such radicals include, but are not limited to, methyl, ethyl, n-propyl, isopropyl, n-butyl, isobutyl, sec-butyl, t-butyl, pentyl, iso-amyl, hexyl, decyl and the like. Such radicals may be substituted with groups other than hydrogen, such as aryl, amino, halogen, cyano, thio (e.g., thioether, sulfhydryl), oxy (e.g., ether, hydroxy), alkoxy, carboxy, oxocarboxy and phosphino.

The term “effective,” as that term is used in the specification and/or claims (e.g., “an effective amount,” means adequate to accomplish a desired, expected, or intended result.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting,” “reducing,” or “prevention,” or any variation of these terms, when used in the claims and/or the specification includes any measurable decrease or complete inhibition to achieve a desired result.

Throughout this application, the term “about” is used to indicate that a value includes the inherent variation of error for the device or the method being employed to determine the value, or the variation that exists among the study subjects. For example, “about” can be within 10%, within 5%, within 1%, or within 0.5%.

The use of the word “a” or “an” or “the” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

When describing the present invention, all terms not defined herein have their common, art-recognized meanings.

It is contemplated that any embodiment discussed in this specification can be implemented with respect to any compound, method, or composition of the invention, and vice versa.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

Cardiac conditions can be either acute or chronic, and either congenital or acquired. The present invention contemplates compounds and methods for the treatment of cardiac conditions. Non-limiting exemplary cardiac conditions include disorders and diseases such as irregular heartbeat disorders and diseases; exercise-induced irregular heartbeat disorders and diseases; sudden cardiac death; exercise-induced sudden cardiac death; congestive heart failure; chronic obstructive pulmonary disease; and high blood pressure. Irregular heartbeat disorders and diseases include and exercise-induced irregular heartbeat disorders and diseases include, but are not limited to, atrial and ventricular arrhythmia; atrial and ventricular fibrillation; atrial and ventricular tachyarrhythmia; atrial and ventricular tachycardia; catecholaminergic polymorphic ventricular tachycardia (CPVT); and exercise-induced variants thereof.

Congestive heart failure, also called congestive cardiac failure or simply heart failure, is a condition that can result from any structural or functional cardiac disorder that impairs the ability of the heart to fill with or pump a sufficient amount of blood throughout the body. Causes and contributing factors to congestive heart failure include the following: genetic family history of heart failure, ischemic heart disease/myocardial infarction, infection, anemia, arrhythmia, hypertension and mitral valve disease.

Cardiac arrhythmia is a group of conditions in which the muscle contraction of the heart is irregular or is faster or slower than normal. Some arrhythmias are life-threatening medical emergencies that can cause cardiac arrest and sudden death. Others cause aggravating symptoms, such as an awareness of a different heartbeat, or palpitation, which can be annoying. Some are quite small and normal. Sinus arrhythmia is the mild acceleration followed by slowing of the normal rhythm that occurs with breathing. In adults the normal heart rate ranges from 60 beats per minute to 100 beats per minute. The normal heart beat is controlled by a small area in the upper chamber of the heart called the sinoatrial node or sinus node. The sinus node contains specialized cells that have spontaneous electrical activity that starts each normal heart beat. A heart rate faster than 100 beats/minute is considered a tachycardia. A serious variety of arrhythmia is known as fibrillation. The muscle cells of the heart normally function together, creating a single contraction when stimulated. Fibrillation occurs when the heart muscle begins a quivering motion due to a disunity in contractile cell function. Fibrillation can affect the atrium (atrial fibrillation) or the ventricle (ventricular fibrillation); ventricular fibrillation is imminently life-threatening.

In alternate examples, the novel compounds generated and described may additionally or alternatively be used to address one or more RyR-associated disorders, diseases, or conditions including cardiac or skeletal muscle conditions, disorders, or diseases. For example, the compounds may be used to reduce the risk of CPVT arrhythmias, (e.g., by targeting one or more of RyR1, RyR2, and RyR3). As another example, the compounds may be used to reduce the risk of ventricular arrhythmias, atrial arrhythmias (such as atrial fibrillation, atrial flutter, etc.), diastolic heart failure, heart failure with reduced ejection fraction, pregnancy-induced cardiomyopathy, hypertrophic cardiomyopathy, dilated cardiomyopathy, skeletal muscle fatigue, and cardiac disease linked to diabetes, and hypertension. In still further examples, the compounds may be used to reduce the risk of malignant hyperthermia, central core disease, heart-stroke, myopathy, diabetes (e.g., diabetic cardiomyopathy, etc.), Duchenne muscular dystrophy, Becker muscular dystrophy, aging-related cognitive dysfunction, chronic obstructive pulmonary disease (COPD), bladder dysfunction, and incontinence.

Abbreviations used herein include SAR—structure activity relationship, COD—cyclic oligomeric depsipeptides, RyR—ryanodine receptor, TPSA—total polar surface area, CASQ2—calsequestrin 2.

Macrocyclic peptide drug candidates have become increasingly popular in the last few decades, largely stemming from their ability to interact with “undruggable” targets. Several macrocyclic, peptidic compounds have already proven to be successful drugs, such as the well-known antibiotic vancomycin, and the immunosuppressant cyclosporine. In this larger size regime (>500 MW), a majority of current macrocyclic drug candidates are natural products or close variants of natural products. Relative to small molecules, there is less flexibility to guide the design of these non-canonical drug candidates to optimize both binding and bioavailability. Studies that further the understanding of macrocyclic peptides and their optimization against biological targets are therefore of great value. Despite these challenges, chemists have made strides to develop non-natural macrocyclic drug candidates, utilizing chemical modifications inspired by nature such as amide N-permethylation, replacement of amides with esters, thioesters/thioamides, or ketones, incorporation of D-amino acids and non-proteinogenic amino acids, side-chain modification, and the use of peptide stapling techniques. Additionally, macrocyclization is increasingly applied to small molecule drug development where single-carbon homologue SAR can be applied to optimize pharmacophore presentation. However, natural products still continue to dominate the examples of macrocycles that are cell permeable, water soluble, and even orally bioavailable. Notable contributions to the development of “drug-like” non-natural macrocycles and metrics to quantify these factors are beginning to emerge, highlighting the importance of these compounds in drug discovery. Peptide and depsipeptide macrocycles particularly, offer an expansive landscape of ring sizes, and the impact of ring size on permeability and solubility is developing slowly.

Considering cyclic depsipeptide natural products, nature appears to leverage ring-size to fine-tune biological activity. Several prominent examples include valinomycin and montanastatin, bassianolide and enniatin C, beauvericin and bassiatin, and the antimycins. Although natural products are ripe with examples of diverse depsipeptides varying in ring size, few studies provide insight to the effect of a systematic variation of ring size and correlation of biological activity at a discrete target. Even then, ring-size is usually only explored as a parameter when the ring-size analogue is also a natural product. The present inventors speculated that the ability to vary only ring size while leaving functionality and other features unchanged might be realized with cyclic oligomeric depsipeptides (CODs).

Due to their oligomeric nature, CODs provide an effective platform to probe ring-size modified pharmacophores wherein size is manipulated while maintaining the functionality inherent to the biologically active molecule. This is an attribute unique to only oligomeric molecules, and something rarely studied in natural product drug design. Examples of the synthesis of non-natural analogues of cyclic depsipeptides include decabassianolide (analogue of bassianolide), a 12-mer and 24-mer of beauvericin, a tetradepsipeptide and cyclohexadepsipeptide analogue of valinomycin, and a 12-mer of bassianolide. More generally, there has been noteworthy work with the synthesis of ring-size analogues in the field of oligolides, as well as cyclic peptides. Yet, still absent from most studies is a large range (>3-mer) of ring sizes, other structural functionality left unchanged, or the evaluation of these compounds against a distinct biological target.

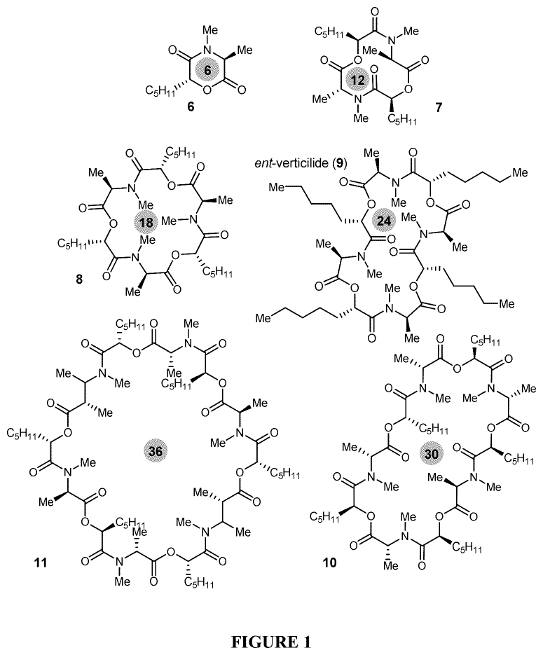

The present inventors reported a study of the fungal COD natural product (−)-nat-verticilide and its enantiomer, (+)-ent-verticilide (9, FIG. 1), to determine their activity against mammalian cardiac ryanodine receptor (RyR2), inspired by the natural product's activity with insect ryanodine receptor. Although the natural product had no effect, the non-natural enantiomer significantly reduced RyR2-mediated spontaneous calcium release. Of the three human isoforms (RyR1, RyR2, and RyR3), RyR2 is predominantly expressed in cardiac SR, and mutations in RyR2 can result in spontaneous calcium release into the SR. This hyperactivity has been indicated in a variety of cardiovascular diseases characterized by ventricular arrhythmias.

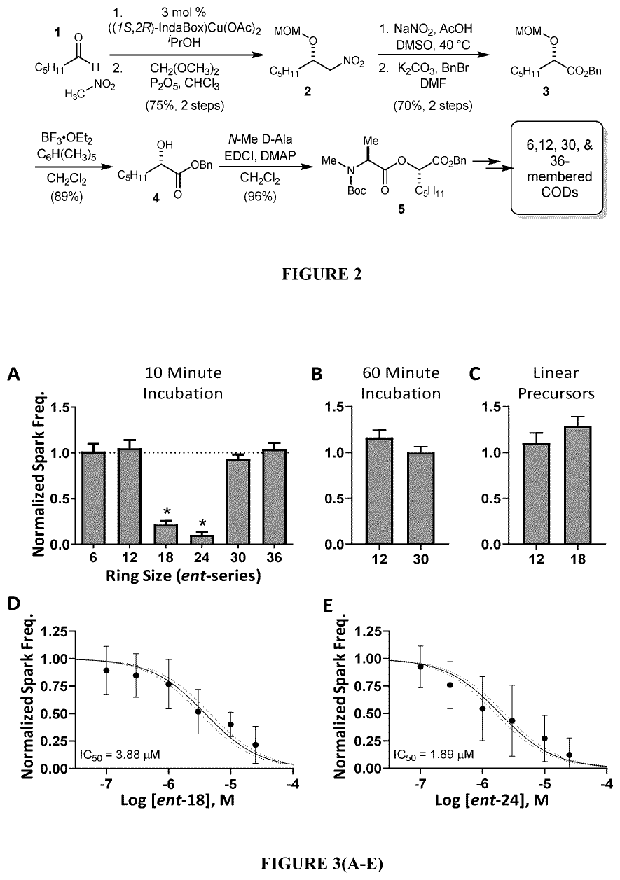

Synthesis of Ring Size Analogues. The present inventors prepared 5 non-natural ring size congeners of ent-verticilide (24-membered COD), including 6-, 12-, 18-, 30-, and 36-membered CODs. ent-Verticilide and the 18-membered COD were prepared using methodology previously reported. The 6-, 12-, 30-, and 36-membered macrocycles were synthesized through a complementary route developed for this purpose (See Scheme 1 (FIG. 2)). The synthesis of the depsipeptide unit begins with an enantioselective Henry reaction using hexanal (1) and nitromethane, followed by MOM protection of the subsequent alcohol. Nitroalkane 2 was then subjected to Mioskowski-Nef conditions to afford the α-hydroxy acid, which was protected as benzyl ester 3. This hydroxy heptanoic ester was then deprotected and coupled with N-methyl-D-alanine to arrive at the key didepsipeptide unit (5). Selective deprotections, followed by condensative couplings provided monomers of varying lengths, which were globally deprotected and cyclized to furnish the CODs of increasing ring size (See Scheme 1).

Activity of Ring-Size Analogues. The activity of the ring size analogues was examined in cardiomyocytes isolated from a CASQ2 gene knockout mouse, a validated model of severe catecholaminergic polymorphic ventricular tachycardia (CPVT) in humans that exhibits pathologically increased RyR2 activity. Ventricular cardiomyocytes were isolated, permeabilized with saponin, and incubated with vehicle (DMSO) or 25 μM of selected compound. RyR2 activity was measured in the form of calcium sparks, which are elementary Ca²⁺ release events generated by spontaneous openings of intracellular RyR2 Ca²⁺ release channels. Importantly, saponin selectively permeabilizes the sarcolemmal membrane, leaving the SR membrane intact and ensuring equivalent access of the compounds to the SR membrane where RyR2 resides.

The compounds were first screened by incubating cardiomyocytes for 10 minutes at 25 μM concentration and compared to vehicle (DMSO) conditions. The 18- and 24-membered rings significantly inhibited RyR2-mediated Ca²⁺ spark frequency, however the 6-, 12-, 30-, and 36-membered rings appeared to have no effect (Error! Reference source not found.A). To ensure there was no delayed binding effect with the inactive rings, longer incubation times for two of the macrocycles were investigated. Extending the incubation time to 60 minutes did not enable any measurable inhibition of spark frequency in either the 12- or 30-membered rings (Error! Reference source not found.3B), suggesting that ring size is a necessary component for activity. Incubation with the 12- or 18-membered linear precursors also demonstrated no inhibition of spark activity—despite clear inhibition with the 18-membered ring—supporting the importance of a cyclic structure for activity (Error! Reference source not found.C). To compare potency, concentration-response curves were generated for the 18- and 24-membered rings. Both compounds inhibited RyR2 with similar potency (Error! Reference source not found.D&E). The natural (D,L) versions of the 6-, 18-, 24-, 30-, and 36-macrocycles were also prepared, but none of these were active in the assay.

Structure-Activity Relationship. Of the five ring-size variants prepared, the 6-, 12-, 30-, and 36-membered ring did not alter calcium spark frequency (Error! Reference source not found.). However, the 18-membered variant (8) was a potent inhibitor. It is important to note, that because the plasma membrane of the cells is permeabilized, cell permeability is removed as a factor contributing to loss of activity. However, several hypotheses for the activity results are proposed by considering the impact of ring size on the conformation and degree of expected rigidity for this ent-verticilide series. The presentation of the methyl and pentyl side chains was expected to deviate significantly in the 6-membered ring variant (6), but as ring-size increases, the conformational mobility is predicted to increase. This is expected to reach a point of limited returns, however, if the COD flexibility begins to work against the tightest binding conformation. This could be the case with the 30- and 36-membered macrocycles. The 6- and 12-membered rings, on the other hand, mirror the difficulties that small molecules display in binding to large, complex targets. The lack of activity from these two ring sizes suggests that ent-verticilide could be interacting with RyR2 over a large surface area, whereas the smallest two ring sizes cannot achieve a similar interaction.

TABLE 1
Comparison of CODs by ring-size, molecular weight, calculated
AlogP values, and calculated topical polar surface area (TPSA).

| | molecular | | calculated |
|---|---|---|---|
| | weight | calculated | TPSA |
| Ring Size | (g/mol) | AlogP^{a} | (Å²)^{b} |
| 6 (6) | 213.27 | 0.95-1.60 | 66.76 |
| 12 (7) | 426.55 | 3.11-3.97 | 100.71 |
| 18 (8) | 669.83 | 4.59-5.19 | 151.69 |
| 24 (9) | 853.11 | 5.05-5.63 | 207.06 |
| 30 (10) | 1066.39 | 5.41-5.83 | 248.17 |
| 36 (11) | 1335.77 | 5.67-5.98 | 303.29 |

^{a}AlogP values were calculated using the Virtual Computational Chemistry Laboratory ALOGPS 2.1 program.
^{b}TPSA values were calculated using QikProp in the Schrödinger software suite.

The present inventors also considered lipophilicity as a second factor. Larger molecular weight compounds can be more lipophilic than their small molecule counterparts. This increase in lipophilicity usually results in a corresponding decrease in aqueous solubility. However, a “chameleon-like” behavior has been attributed to macrocyclic compounds, describing their ability to adopt conformations that bury hydrophilic or hydrophobic functionality based on their environment. This attribute of molecules well outside of the traditional realm of drug-likeness endows them with both the lipophilicity needed to cross cell membranes, as well as aqueous solubility. These two properties are crucial for activity with intracellular targets along with bioavailability. In this ring-size series, both the A log P values and the topical polar surface areas (TPSA) were calculated to quantify the relative lipophilicity for each compound. These calculated values follow the expected trend—as the ring-size increases, lipophilicity increases (Table 1). In these initial studies, however, a corresponding decrease in solubility was not observed. Each ring-size was soluble in the aqueous buffer used in the calcium sparks assays at 25 μM, and no complications with precipitation were observed. This was further verified by kinetic solubility studies (see supporting information). Additionally, ent-verticilide maintains activity in vivo, indicating some level of aqueous solubility. This increase in lipophilicity could, however, still be a factor contributing to the loss of activity. If the target binding surface includes numerous hydrogen bonding sites, the pharmacophore could have surpassed a non-polar threshold in the larger macrocycles. Furthermore, increases in lipophilicity could reduce the target specificity, resulting in a loss of activity through off-target binding. The smaller ring sizes (6- and 12-membered rings) may suffer from increased rigidity, an effect noted in studies of a decapeptide analogue of cyclosporine.

Unexpectedly, the present inventors discovered another potent ring size variant—the 18-membered COD. This finding indicates that the full structure of ent-verticilide, a 24-membered cyclic oligomeric depsipeptide, is not critical to its activity. Without being bond by theory or mechanism, these data support an intriguing hypothesis where a region, rather than total volume of the molecule is responsible for its activity. It also indicates that conformation plays an essential role, as the 12-membered ring and the 12-membered linear precursor, exactly “half” variants of ent-verticilide, display no activity.

In one embodiment, this invention relates to compounds and pharmaceutically acceptable derivatives thereof.

One aspect relates to a compound of the following formula:

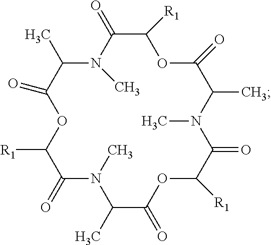

wherein each R₁ is independent and chosen from C₁-C₁₂ substituted or unsubstituted alkyl; and pharmaceutically acceptable salts thereof.

Another aspect relates to compounds where R₁ is C₁-C₆ alkyl. Another relates to compounds where R₁ is C₁-C₅ alkyl. Yet another relates to where R₁ is C₅H₁₁.

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids” includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a

pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention can comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

It is understood that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing The disclosed compounds may be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) may be administered by a route and in an amount commonly used therefore, contemporaneously or sequentially with a disclosed compound. When a disclosed compound is used contemporaneously with one or more other drugs, a pharmaceutical composition in unit dosage form containing such drugs and the compound is preferred. However, the combination therapy can also be administered on overlapping schedules. It is also envisioned that the combination of one or more active ingredients and a disclosed compound can be more efficacious than either as a single agent.

In one aspect, the compounds can be co-administered with a known anti-arrhythmic. In another aspect, the compound can be co-administered with a known beta-blocker.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

As indicated above, as used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

### EXAMPLES

### Example 1

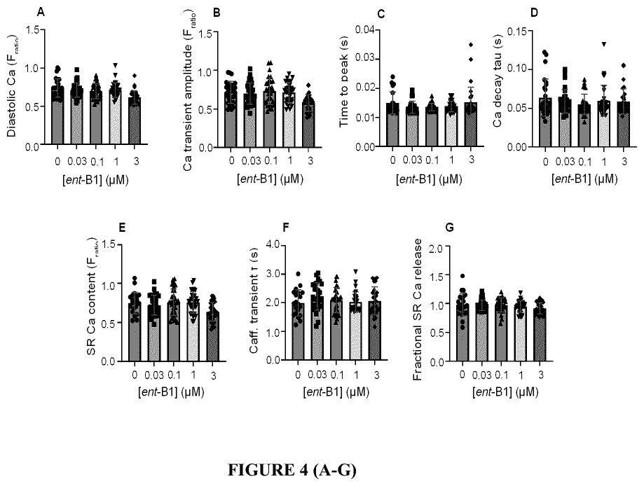

This example shows intracellular Ca²⁺ measurements from intact Casq2^{-/-} cardiomyocytes. FIG. 4(A) shows diastolic Ca²⁺ measured during field stimulation. FIG. 4(B) shows paced Ca²⁺ transient amplitudes. FIG. 4(C) shows time to peak and (D) Ca²⁺ decay kinetics of paced Ca²⁺ transients. FIG. 4(E) shows caffeine-induced Ca²⁺ transient amplitude. FIG. 4(F) shows caffeine-induced Ca²⁺ transient decay kinetics. FIG. 4(G) shows fractional Ca²⁺ release (paced transient amplitude/SR Ca²⁺ content). For panels A-D and G, N=26, 30, 30, 30, and 31 cells for DMSO, 0.03, 0.1, 1, and 3 μM ent-B1, respectively. For panels E and F, N=14, 27, 29, 29, and 30 cells for DMSO, 0.03, 0.1, 1, and 3 μM ent-B1, respectively. Data are shown as mean±SD with individual data points.

### Example 2

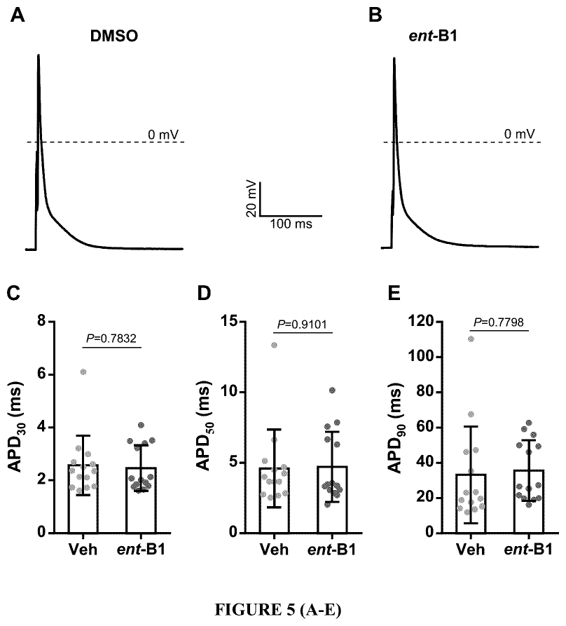

This example shows action potential measurements in intact Casq2^{-/-} ventricular myocytes. FIG. 5(A-B) Representative membrane potential recordings from current-clamped myocytes stimulated at 1 Hz. Myocytes were pre-incubated for 2½ hrs with vehicle (DMSO) (A) or 0.3 μM ent-B1 (B) before patch clamping individual myocytes. Dotted line indicates 0 mV. FIG. 5(C-E) Summary of action potential durations (APD_{30, 50 & 90}). N=14 cells from 2 Casq2^{-/-} mice each. Individual data points are shown with mean±SD. All data were analyzed using unpaired t test.

### Example 3

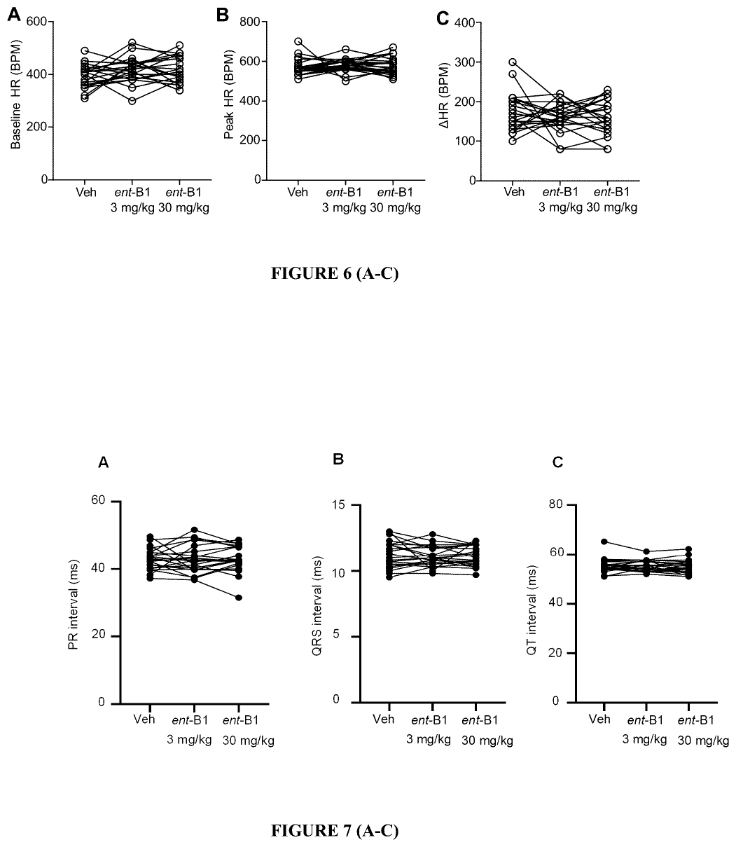

This example shows the effect of ent-B1 on heart rate of Casq2^{-/-} mice. Mice were given vehicle (DMSO) or ent-B1 by intraperitoneal injection 15 min before isoproterenol challenge. FIG. 6(A) shows baseline heart rate (beats per minute). FIG. 6(B) shows peak heart rate and FIG. 6(C) shows change in heart rate following intraperitoneal administration of 3 mg/kg isoproterenol. Mixed effects model pre-test P>0.05 for period, sequence, and treatment in all groups in each panel.

### Example 4

This example shows the effect of ent-B1 on electrocardiogram (ECG) parameters of Casq2^{-/-} mice. Mice were given vehicle (DMSO) or ent-B1 by intraperitoneal injection 15 min before isoproterenol challenge. FIG. 7(A) PR, FIG. 7(B) QRS, and FIG. 7(C) show QT intervals were measured during baseline ECG recording 2 minutes prior to administration of 3 mg/kg isoproterenol. Mixed effects model pre-test P>0.05 for period, sequence, and treatment in all groups in each panel.

### Example 5

This example shows the in vitro pharmacology of an exemplary compound of the present invention through in vivo experiments in an established CPVT mouse model (Casq2^{-/-} mice).

### Material and Methods

Drugs, chemicals, and reagents—All chemicals and reagents were purchased from Sigma-Aldrich unless otherwise stated. A compound of the present invention, ent-B1, was synthesized.

Single Channel Recording—SR vesicles containing RyR2 were isolated from porcine hearts and incorporated in artificial bilayer membranes. Lipid bilayers were formed across an aperture with diameter 150-250 mm of a delrin cup using a lipid mixture of phosphatidylethanolamine, phosphatidylserine, and phosphatidylcholine (5:3:2 wt/wt, Avanti Polar Lipids, Alabaster, AL) in n-decane (50 mg/ml, ICN Biomedicals, Irvine, CA). During the SR vesicle fusion period, the cis (cytoplasmic) chamber contained 250 mM Cs⁺ (230 mM CsCH₃O₃S, 20 mM CsCl)+1.0 mM CaCl₂ and the trans (luminal) chamber contained 50 mM Cs⁺ (30 mM CsCH₃O₃S, 20 mM CsCl)+1 mM CaCl₂. When ion channels were detected in the bilayer, the trans Cs⁺ was raised to 250 mM by aliquot addition of 4 M CsCH₃O₃S. During experiments, the cis solution was exchanged by a perfusion system to one containing 250 mM Cs⁺ plus 2 mM ATP and free Ca²⁺ of 100 nM followed by exchange with the same plus ent-B1. Thus, the perfusion system allowed repeated application and washout of ent-B1 within ˜3 s.

All solutions were pH buffered using 10 mM TES (N-tris[hydroxymethyl] methyl-2-aminoethanesulfonic acid; ICN Biomedicals) and titrated to pH 7.4 using CsOH (ICN Biomedicals). Free Ca²⁺ of 100 nM was generated from 1 mM CaCl₂ and 4.5 mM BAPTA (1,2-bis(o-aminophenoxy)ethane-N,N,N′,N′-tetraacetic acid; obtained from Invitrogen) and this was validated using a Ca²⁺ electrode. ATP was in the form of the di-sodium salt and obtained from Enzo Life Sciences (Farmingdale, NY) and Cs⁺ salts were obtained from Sigma-Aldrich (St Louis, MO). CaCl₂ was obtained from BDH Chemicals (VWR, Radnor, PA). Cytoplasmic recording solutions were buffered to a redox potential of −232 mV with glutathione disulfide (GSSG; 0.2 mM) and glutathione (GSH; 4 mM; MP Biomedicals), and luminal solutions were buffered to a redox potential of −180 mV with GSSG (3 mM) and GSH (2 mM), both obtained from MP Biomedicals. ent-B1 was prepared as a stock solution in DMSO.

Single Channel Recording Analysis and Data Acquisition—Experiments were carried out at room temperature (23±2° C.). Electric potentials are expressed using standard physiological convention (i.e. cytoplasm relative to SR lumen at virtual ground). Control of the bilayer potential and recording of unitary currents was done using an Axopatch 200B amplifier (Axon Instruments/Molecular Devices, Sunnyvale, CA). Channel currents were digitized at 50 kHz and low pass filtered at 5 kHz. Before analysis the current signal was redigitized at 5 kHz and low pass-filtered at 1 KHz. Individual readings of open probability were derived from 30-60 s of RyR2 recording. Single channel open probability was measured using a threshold discriminator at 50% of channel amplitude.

[³H]ryanodine ligand binding assay—[³H]ryanodine, [9,21-3H(N)] (56 Ci/mmol) was obtained from PerkinElmer. Porcine cardiac SR vesicles were isolated as previously described (Fruen et al., 2000) and incubated with, 200 nM CaM binding peptide, 0.1 μM CaCl2, 20 mM PIPES, 150 mM KCl, 5 mM GSH, 0.1 mg/mL BSA, 1 μg/mL aprotininin, lug/mL leupeptin, and 1 μM DTT for 30 min at 37° C. Samples were centrifuged at 110,000×g for 25 min at 4° C. and resuspended to at final concentration of 15 mg/mL in 20 mM PIPES, 150 mM KCl, 5 mM GSH, 0.1 mg/mL BSA, 1 μg/mL aprotinin, 1 μg/mL leupeptin, and 1 μM DTT. In 96-well plates, cardiac SR membranes (CSR, 3 mg/mL) were pre-incubated with 1% v/v ent-B1 (to yield the indicated drug concentrations) for 30 min, at 22° C., in a solution containing 150 mM KCl, 5 mM GSH, 1 μg/mL Aprotinin/Leupeptin, 1 mM EGTA, and 23 μM or 1.62 mM CaCl₂ (100 nM or 30 μM free Ca²⁺, respectively as determined by MaxChelator), 0.1 mg/mL BSA, and 20 mM K-PIPES (pH 7.0). Non-specific [³H]ryanodine binding to SR was assessed by addition of 15 μM non-radioactive ryanodine. Maximal [³H]ryanodine binding was assessed by addition of 5 mM adenylyl-imidodiphosphate (AMP-PNP), supplemented with 20 mM caffeine. These control samples were each loaded over four wells per plate. Binding of [³H]ryanodine (7.5 nM) was determined following a 3 h incubation at 37° C. and filtration through grade GF/B glass microfiber filters (Brandel Inc., Gaithersburg, MD, US) using a M96T-Brandel Harvester. Filters were immersed in 4 mL of Ecolite scintillation cocktail and incubated 24 hours prior to [³H] counting in a Perkin-Elmer Tri-Carb 4810.

Intracellular Ca²⁺ measurements in intact cardiomyocytes—Ventricular cardiomyocytes were isolated from two male 10-week old and one female 13-week old Casq2^{-/-} mouse as described previously (Knollmann et al., 2006). Cardiomyocytes were pre-incubated for 2 hours with DMSO or ent-B1. Myocytes were then loaded with Fura-2 acetoxymethyl ester (Fura-2 AM; Invitrogen) as described previously (Batiste et al., 2019). Briefly, isolated single ventricular myocytes were incubated with 2 μM Fura-2 AM for 7 minutes to load the indicator in the cytosol. Myocytes were then washed twice for 10 minutes with normal Tyrode (NT) solution containing 250 μM probenecid (all solutions also contained either vehicle [DMSO] or ent-B1). The composition of NT used for Fura-2 loading and washing was (in mM): 134 NaCl, 5.4 KCl, 1.2 CaCl2, 1 MgCl2, 10 glucose, and 10 HEPES, pH adjusted to 7.4 with NaOH. After Fura-2 loading, all experiments were conducted in NT solution containing 1 μM isoproterenol and 2 mM CaCl2. Fura2-loaded myocytes were electrically paced at 3 Hz field stimulation and Ca transients were recorded for 20 seconds followed by no electrical stimulation for 40 seconds to record spontaneous Ca release events. After that, myocytes were perfused with 10 mM caffeine in NT solution for 5 seconds to estimate total SR Ca content. Fura-2 was measured using a dual-beam excitation fluorescence photometry setup (IonOptix Corp.) and analyzed using commercially available data analysis software (IonWizard, IonOptix, Milton, MA). All experiments were conducted at room temperature.

Action potential measurements in intact cardiomyocytes—Membrane potential was recorded in current-clamp mode with ruptured patch in isolated ventricular myocytes from one male 13-week old and one female 11-week old Casq2^{-/-} mouse. To match the drug incubation time of the intracellular Ca measurements, myocytes were pre-incubated in vehicle (DMSO) or 0.3 μM ent-B1 for 2.5 hrs before AP measurement. Action potential (APs) were measured using pipette solutions containing (in mmol/L) 120 K-aspartate, 20 KCl, 5 NaCl, 5 MgATP, 0.1 EGTA and 10 HEPES adjusted to pH 7.2 with KOH. Whole-cell patch was established in perfused Tyrode solution containing (in mmol/L) 134 NaCl, 5.4 KCL, 1 MgCl₂, 2 CaCl₂, 10 glucose and 10 HEPES and pH adjusted to 7.4 with NaOH. All Tyrode solution contained DMSO or 0.3 μM ent-B1. APs were triggered by application of a 2 ms current injection at 20-30% above threshold with 1 Hz and obtained using Multiclamp 700B, Digidata 1550B. APDs were analyzed using pClamp 10.6 software (Axon Instruments, CA, USA). All experiments were conducted at room temperature.

In vivo pharmacokinetic study—The pharmacokinetic study was carried out by a contract research organization, Pharmaron, Inc. Mice were housed with free access to food and water. All protocols and procedures were compliant with Animal care and Use Application approved by the Institution Animal Care and Use Committee of Pharmaron, Inc., (protocol #PH-DMPK-VUMC-22-003) following the guidance of the Association for Assessment and Accreditation of Laboratory Animal Care (AAALAC). An ent-B1 dose of 3 mg/kg (drug/body weight) was used. Three male CDI mice, age 6-8 weeks, were selected for the study. Mice were injected intraperitoneally with ent-B1 dissolved in solution containing 10% Tween 20, 10% DMSO, 40% water, and 40% PEG-400 (v/v, 5 mL/kg), with a final concentration of 0.6 mg/mL. 30 μL of blood was collected from each animal at 10, 20, 30, 60, 180, and 480 minutes following drug administration and centrifuged at 5000×g, 4° C. for 5 minutes to obtain plasma. The samples were stored at −75±15° C. until analysis. Clinical observation showed no abnormality during the entire experiment.

LC-MS/MS analysis of ent-B1—LC-MS/MS with electronspray ionization in the positive ion mode setting was used to detect ent-B1 followed by multiple reaction monitoring of precursor and product ions as follows: ent-B1 (mass-to-charge ratio [m/z] 640.18 to 214.00). Mouse plasma was quantified using nine standards (0.5-1000 ng/ml) and four quality control levels (1, 2, 50, 800 ng/ml) prepared independently of those used for the standard curve. 20 μL of standards, quality control samples, and unknown samples (10 μL plasma and 10 μL blank solution) were added to 200 μL acetonitrile containing internal standard (dexamethasone; [m/z] 393.40 to 373.30) for protein precipitation. The samples were vortexed for 30 seconds, centrifuged for 15 minutes at 4000 rpm and 4 [symbol] ° C., and the supernatant was diluted five times with water. Samples were then loaded into SIL-30AC autosampler and 10 μL was injected into a Shimadzu LC-30AD Series HPLC coupled to an AB Sciex Triple Quad 5500 mass spectrometer. Analytes were separated on a Raptor Biphenyl column (50×2.1 mm, 2.7 um) using a 95:5 (v/v) mobile phase mixture of 0.1% formic acid in water (mobile phase A) and 0.1% formic acid in acetonitrile (mobile phase B) at the flow rate of 0.6 ml/min. The gradient for mobile phase B was increased to 95% over 1.4 minutes with a total run time of 2.5 minutes for each sample. All quality controls and standards met the following acceptance criteria 1) standard curve of at least five standards are within 15% of their nominal concentrations and at least 50% at each quality control level (low, medium, and high) were within 15% of their nominal concentrations.

Pharmacokinetic analysis—Plasma concentrations of ent-B1 at each time point were imported to Phoenix WinNonlin® 8.0 software (Certara USA, Inc., Princeton, NJ). Plasma concentration time profiles for individual animals were analyzed by noncompartmental analysis using model 200 (Plasma; Single Extravascular Dose; Linear Log Trapezoidal Method) to approximate the elimination rate constant (ke), half-life (T_{1/2}), maximum observed plasma concentration (Cₘₐₓ), time to maximum observed plasma concentration (Tₘₐₓ), the area under the plasma concentration-time curve from zero to infinity (AUC_{inf}). Dose was normalized to 3 mg/kg for each animal and used to derive estimates of extravascular clearance (Cl/F) and extravascular volume of distribution (Vz/F) by noncompartmental analysis.

Plasma stability experiments—Drug-free mouse plasma was preincubated in a 96-well polypropylene plate for 30 minutes on a microplate shaker at 37° C. prior to the addition of ent-B1 (final concentration 1900 ng/mL). Drug was added to individual wells (n=3 per compound), mixed thoroughly, and 50 μL of plasma was removed at 0, 15, 30, 45 60, 90, and 120 minutes to evaluate compound stability in mouse plasma at 37° C. Samples removed at each time point were transferred to a 96-well polypropylene plate containing 300 μL of ice-cold acetonitrile containing internal standard (ent-verticilide-d₁₂; 100 ng/mL). Samples were mixed multiple times with a pipette to ensure precipitation of plasma proteins and the plate was covered with an adhesive film to prevent solvent evaporation. Samples remained on ice throughout the experiment until the last sample collection time point. The plate was centrifuged at 4800 rpm for 20 minutes and 300 μL of supernatant was transferred to a new 96 deep well polypropylene deep well plate, evaporated under a stream of nitrogen gas, and reconstituted with 200 μL of mobile phase prior to LCMS analysis. Peak area ratios of the analyte and internal standard for each sample were analyzed by liquid chromatography coupled to tandem mass spectrometry (LC-MS/MS) and normalized to the area ratio for time point 0 (baseline) to calculate the percent of ent-B1 remaining at each time point. Criteria for establishing compound stability was set at 90% and 80% remaining at 60 and 120 minutes, respectively.

ECG recording—Nine male and thirteen female Casq2^{-/-} mice 17-20 weeks old were randomly assigned to a three-by-three crossover design such that every treatment sequence was sampled. Mice were pretreated with intraperitoneal injection of vehicle (DMSO), 3 mg/kg ent-B1, or 30 mg/kg of ent-B1 fifteen minutes prior to baseline ECG recording. Mice were then injected with 3 mg/kg isoproterenol intraperitoneally. Recordings were continued for 5 minutes or for 1 minute after cessation of ventricular ectopy. A washout period of one week was used between treatments. The Vanderbilt Institutional Animal Care and Use Committee (IACUC) approved the use of mice in these studies (protocol #M1900057-01). Animals had free access to food and water.

ECG analysis—LabChart (AD Instruments, Inc) was used to analyze ECG recordings by a reviewer blinded to treatment dose. ECG records were examined to quantify premature ventricular contractions (PVCs), duration of ventricular tachycardia, heart rate, and baseline ECG parameters (PR, QRS and QT interval). Arrhythmias were scored on a five point ordinal scale based on the number of PVCs with the following criteria: 1) zero point for no PVCs; 2) one point for isolated PVCs; 3) two points for bigeminy (alternating sinus beats and PVCs); 4) three points for couplets (two consecutive PVCs); and 5) four points for three or more consecutive PVCs (ventricular tachycardia).

Statistical Analysis—Statistics were carried out in GraphPad Prism (v9.5.0) or Matlab as indicated in the figure legends. Notably, given the known in vitro and in vivo efficacy of ent-1—a ring size analogue to ent-B1—we conducted exploratory studies of ent-B1 without a prespecified null hypothesis. Hence, all p-values should be interpreted as descriptive and not hypothesis-testing. All tests were two-sided and a p-value cutoff of <0.05 was used to denote significance. Concentration-response curves for RyR2 single-channel, [³H]ryanodine ligand binding and intact myocyte assays were generated using non-linear regression with the equation: Y=Bottom+(Top−Bottom)/(1+(IC₅₀/X)\hat{ }HillSlope) using least square regression, no weighting, and no constrains on the parameters. The distributions of relative RyR2 open probability at each [ent-B1] were normalized by taking the log of each sample. For the 3×3 crossover in vivo ECG study, a pre-test was conducted using a mixed model with fixed effects of sequence and period; mice were treated as random effects. When warranted, a post-hoc test was conducted and used, as reported in the pertaining figure legends, to test the null hypothesis that the 3 mg/kg or 30 mg/kg doses do not deviate from treatment with vehicle.

### Results

### Ent-B1 Inhibits RyR2 Single Channels and [³H]ryanodine Binding in SR Vesicles

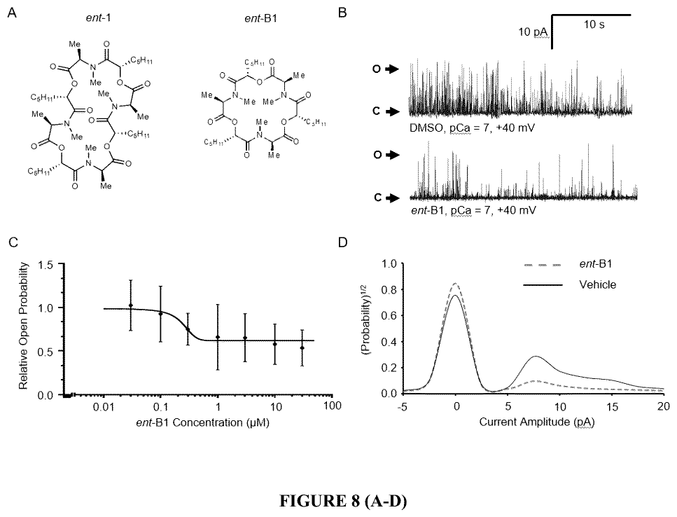

To determine the effect of ent-B1 on RyR2 channel activity, the inventors studied isolated SR vesicles derived from porcine cardiac muscle. RyR2 channels were incorporated into artificial lipid bilayers and exposed to ent-B1 via perfusion (FIG. 8). We tested the effect of seven concentrations of ent-B1 (0.03, 0.1, 0.3, 1, 3, 10, and 30 μM) on RyR2 channel open probability. Cytosolic Ca²⁺ concentration was kept at 100 nM to match physiological concentrations during diastole. ent-B1 inhibited RyR2 channels in a concentration-dependent manner, with an estimated IC₅₀ of ˜0.24 μM [95% confidence interval (CI): 0.08-1.00] and incomplete maximal channel inhibition (FIG. 8C). There was a corresponding shift in the current amplitude histogram toward I=0 in the presence of ent-B1, with no detectable substates. (FIG. 7D). To establish a more detailed concentration-response relationship, the present inventors performed a [³H]ryanodine binding assay using SR samples from porcine cardiac muscle. In this assay of RyR2 channel activity, the presence of an inhibitor reduces the amount of [³H]ryanodine bound to the samples. When compared to vehicle (DMSO), ent-B1 reduced [³H]ryanodine binding with a low micromolar potency (IC₅₀=1.9 μM) and incomplete maximal inhibition of approximately 30% (see FIG. 10). Taken together, these results demonstrate that ent-B1, a compound of the present invention, directly inhibits RyR2 channels.

### Ent-B1 Reduces Spontaneous Ca²⁺ Release in Intact Mouse CPVT Cardiomyocytes

Direct pharmacological block of RyR2 necessitates a compound permeate the cell membrane (sarcolemma). Single channel and [³H]ryanodine assays do not require cell membrane permeability or transport. Hence, the present inventors examined the activity of ent-B1 in cardiomyocytes isolated from Casq2^{-/-} mice to determine whether ent-B1 blocks RyR2 in intact cells. To quantify drug efficacy, the inventors measured the rate of spontaneous RyR2-mediated spontaneous SR Ca²⁺ release events following a 3 Hz pacing protocol (FIG. 10). Compared to vehicle (DMSO), ent-B1 reduced the rate of SCRs in a concentration-dependent manner (IC₅₀=0.23±0.1 μM) with ˜60% efficacy at the highest concentration tested (FIG. 10B). This indicates that after 2.5 incubation, ent-B1 significantly inhibits pathologic Ca²⁺ release in intact cardiomyocytes with a similar potency as in the RyR2 single channel assay (0.24 μM, FIG. 8). Only the highest concentration of ent-B1 tested (3 μM) reduced diastolic Ca²⁺ levels, the amplitude of the systolic Ca²⁺ transient, and the amplitude of the caffeine-induced Ca²⁺ transient, which is consistent with a mechanism of action of a partial RyR2 inhibitor. ent-B1 had no effect on other measures of SR Ca²⁺ release such as time-to-peak of Ca²⁺ transients or Ca²⁺ decay kinetics, the latter being an indicator of SERCA2 Ca²⁺ SR uptake rate.

### Ent-B1 is Stable in Plasma and has Druggable Pharmacokinetic Properties

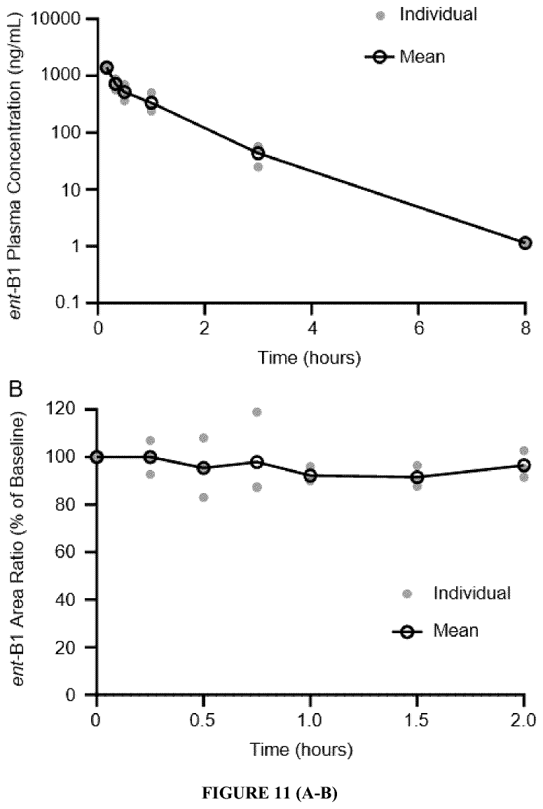

Once it was established that ent-B1 could directly bind to RyR2 and permeate the sarcolemma to inhibit pathologic Ca²⁺ release, the present inventors wanted to test its therapeutic efficacy in an in vivo arrhythmia study. To guide dose selection, the inventors first determined ent-B1's pharmacokinetic properties in mice. Plasma samples were collected and ent-B1 concentrations measured after a 3 mg/kg ent-B1 i.p. injection. Substantial plasma concentrations were readily achieved, indicating favorable systemic exposure after i.p. administration. The mean peak plasma concentration (Cₘₐₓ) was 1460 ng/ml (2.3 μM) at 10 min after i.p. injection and exhibited a biphasic decline with a mean elimination half-life (t_{1/2}) of 45.4 minutes (See FIG. 11A and Table 2). The half-life for ent-B1 was substantially shorter than the half-life of its larger ring size analogue, ent-1, which was 6.9 and 6.4 hours for the 3 mg/kg and 30 mg/kg doses, respectively. To determine whether the shorter half-life of ent-B1 was due to instability of the drug in plasma, the inventors used LC-MS/MS to measure ent-B1 incubated in murine plasma. ent-B1 was stable over the course of two hours, with ˜95% of the parent compound remaining (FIG. 11B).

### Ent-B1 Reduces Ventricular Arrhythmia Burden in a Mouse Model of CPVT

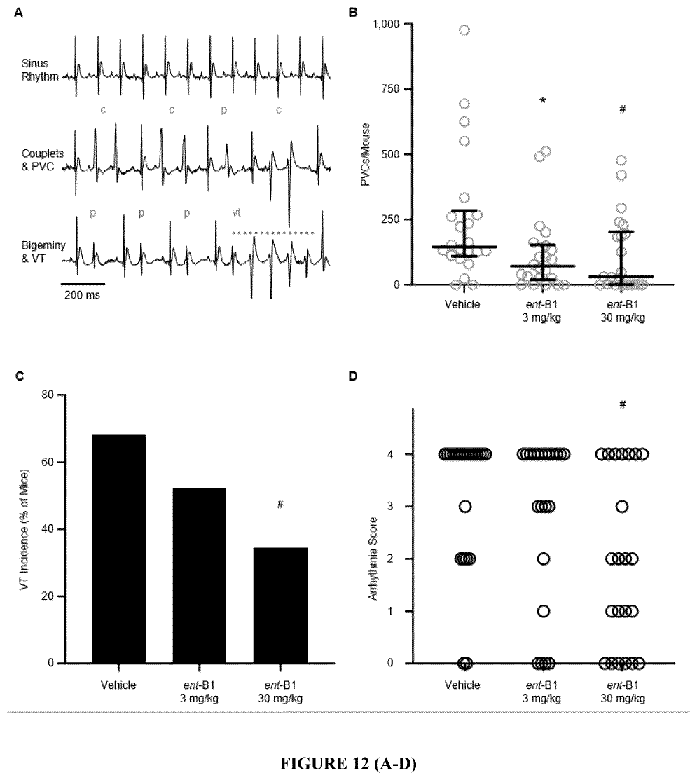

Casq2^{-/-} mice are a validated model for preclinical testing of antiarrhythmic drugs for CPVT. The exemplary compound ent-1 had antiarrhythmic efficacy in vivo in a single dose study at 3 and 30 mg/kg i.p. in Casq2^{-/-} mice. The same doses of ent-B1 were tested in a triple-crossover design with each mouse receiving vehicle (DMSO), 3 mg/kg, and 30 mg/kg with a one-week washout period between experiments. Based on the pharmacokinetic properties of ent-B1 (Table 2, below), an in vivo arrhythmia challenge protocol was chosen with the data collection occurring 15 minutes after ent-B1 i.p. administration. Mice were anesthetized with isoflurane and a baseline electrocardiogram (ECG) was established. Ventricular arrhythmias were elicited with a catecholamine challenge using the β-adrenergic agonist isoproterenol (3 mg/kg i.p.). ECGs were analyzed in blinded fashion and ventricular arrhythmias quantified. FIG. 12A gives examples of ventricular arrhythmias induced by the catecholamine challenge. ent-B1 caused a dose-dependent reduction in the number of total ectopic beats and incidence of VT (FIG. 12B, C). We also scored the mice based on the ventricular arrhythmia severity, i.e., premature ventricular complexes (PVCs)<bigeminy<couplets<ventricular tachycardia. The lower arrhythmia risk scores in mice receiving ent-B1 indicate therapeutic efficacy (FIG. 12D).

TABLE 2
Noncompartmental pharmacokinetic (PK) parameter estimates of ent-B1
following 3 mg/kg intraperitoneal administration in CD1 mice

| PK parameter | 3 mg/kg |
|---|---|
| ke (h−1) | 0.932 ± 0.139 |
| T_{1/2} (h) | 0.756 ± 0.123 |
| Tₘₐₓ (h) | 0.167 ± 0.0 |
| Cₘₐₓ (ng/ml) | 1460 ± 90 |
| AUC_{inf} (h × ng/ml) | 1020 ± 184 |
| Vz/F (L/kg) | 3.32 ± 1.00 |
| Cl/F (L/h/kg) | 3.01 ± 0.50 |

ke: elimination rate constant; T_{1/2}: elimination half-life; Tₘₐₓ: time to maximum concentration; Cₘₐₓ: maximum concentration; AUC_{inf}: area under the plasma concentration curve from zero to infinity time; Vz/F: extravascular volume of distribution; Cl/F: extravascular clearance. Data reported as mean ± standard deviation.

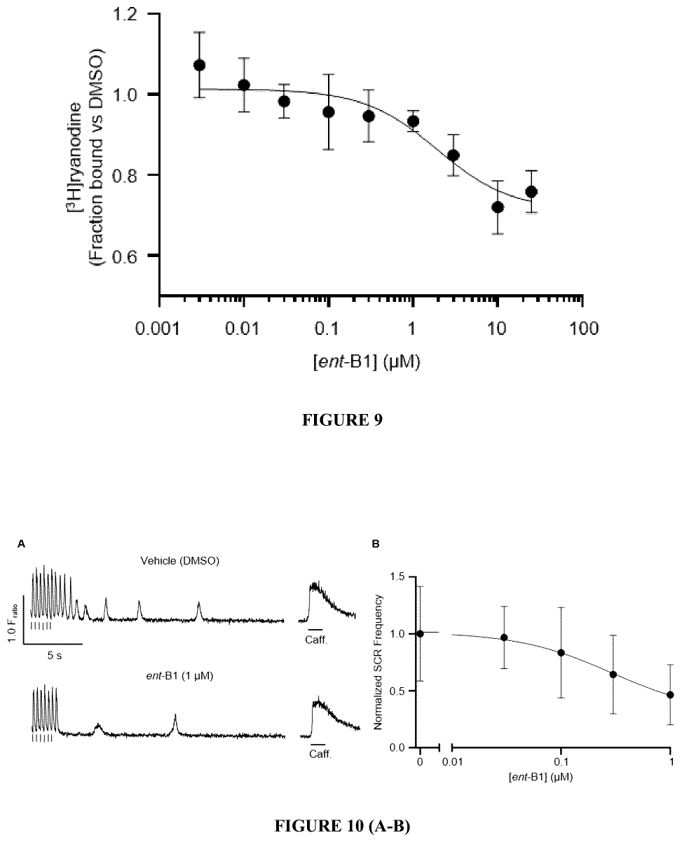

In vitro experiments suggest that a compound of the present invention, ent-B1 has a slightly lower potency than the larger ring-size analogue ent-1. For example, the IC₅₀ for ent-1 in the ryanodine binding assay was 0.1 μM, whereas ent-B1 was 1.3 μM (See FIG. 9). In the intact cardiomyocyte assay, the difference in potency was less, with an IC₅₀ of 0.09 μM for ent-1, and 0.23 μM for ent-B1 (See FIG. 10). Importantly, the ent-B1 potency in the most direct assay, the RyR2 single channel assay, was 0.24 μM (FIG. 7), like the intact myocyte assay (FIG. 9). However, the in vivo potency of ent-B1 on ventricular arrhythmia suppression was comparable to that of ent-1, with both compounds dosed at 30 mg/kg producing a 70% reduction in ventricular ectopy burden using the same arrhythmia induction protocol (see FIG. 12B). Also observed is a clear dose-dependent effect of ent-B1 in vivo (FIG. 12). At the same time, ventricular ectopy was not completely suppressed even though peak plasma concentrations in vivo were more than 10-fold higher than the IC₅₀ in cardiomyocytes. The present inventors attribute this result to the incomplete inhibition of RyR2 observed in vitro, although high plasma protein binding and/or low membrane permeability could be contributory. It should be noted that a statistically significant suppression of ventricular tachycardia, the most clinically-relevant arrhythmia parameter, was observed only at the higher dose tested (30 mg/kg, FIG. 12). The present invention shows that compounds of the present invention have antiarrhythmic efficacy in the most physiologically relevant assay, the in vivo arrhythmia challenge.

In vitro assays suggest that, like ent-1, ent-B1 directly binds to RyR2 and produces incomplete inhibition. Without being bound by theory or mechanism, these data suggest that ent-B1 likely acts as a negative allosteric modulator of channel function, similar to ent-1, for which the inventors identified a putative binding site. ent-B1's combination of sub-micromolar potency and partial channel inhibition is ideal for a target, as a stronger inhibition of RyR2 could be fatal given its central role in physiologic excitation-contraction coupling. Although peak plasma concentration was comparable to ent-1, pharmacokinetic studies revealed a more rapid systemic clearance of ent-B1 (t_{1/2}=45 min, table 2) relative to ent-1 (415 min) in mice. Given the short half-life, ent-B1 would be particularly advantageous in acute clinical scenarios. For example, compounds of the present invention are particularly useful for treatment of ventricular tachycardia storm (“VT storm”), an acute, life-threatening arrhythmia disorder consisting of sequential episodes of sustained VT in a 24-hour window.

### REFERENCES

Batiste S M, Blackwell D J, Kim K, Kryshtal D O, Gomez-Hurtado N, Rebbeck R T, Cornea R L, Johnston J N and Knollmann BC (2019) Unnatural verticilide enantiomer inhibits type 2 ryanodine receptor-mediated calcium leak and is antiarrhythmic. Proc Natl Acad Sci USA 116(11): 4810-4815.
Bers D M (2002) Cardiac excitation-contraction coupling. Nature 415(6868): 198-205.
Blackwell D J, Faggioni M, Wleklinski M J, Gomez-Hurtado N, Venkataraman R, Gibbs C E, Baudenbacher F J, Gong S, Fishman G I, Boyle P M, Pfeifer K and Knollmann B C (2022) The Purkinje-myocardial junction is the anatomic origin of ventricular arrhythmia in CPVT. JCI Insight 7(3).
Blackwell D J, Smith A N, Do T, Gochman A, Schmeckpeper J, Hopkins C R, Akers W S, Johnston J N and Knollmann B C (2023) In Vivo Pharmacokinetic and Pharmacodynamic Properties of the Antiarrhythmic Molecule ent-Verticilide. J Pharmacol Exp Ther 385(3): 205-213.
Brunton L L and Knollmann B C (2023) Goodman & Gilman's the pharmacological basis of therapeutics. Fourteenth edition. ed. McGraw Hill Medical, New York.
Cardiac Arrhythmia Suppression Trial I (1989) Preliminary report: effect of encainide and flecainide on mortality in a randomized trial of arrhythmia suppression after myocardial infarction. N Engl J Med 321(6): 406-412.
Caron G, Kihlberg J, Goetz G, Ratkova E, Poongavanam V and Ermondi G (2021) Steering New Drug Discovery Campaigns: Permeability, Solubility, and Physicochemical Properties in the bRo5 Chemical Space. ACS Med Chem Lett 12(1): 13-23.
Chopra N, Kannankeril P J, Yang T, Hlaing T, Holinstat I, Ettensohn K, Pfeifer K, Akin B, Jones L R, Franzini-Armstrong C and Knollmann B C (2007) Modest reductions of cardiac calsequestrin increase sarcoplasmic reticulum Ca2+ leak independent of luminal Ca2+ and trigger ventricular arrhythmias in mice. Circ Res 101(6): 617-626.
Giannini G, Conti A, Mammarella S, Scrobogna M and Sorrentino V (1995) The ryanodine receptor/calcium channel genes are widely and differentially expressed in murine brain and peripheral tissues. J Cell Biol 128(5): 893-904.
Kim K, Blackwell D J, Yuen S L, Thorpe M P, Johnston J N, Cornea R L and Knollmann B C (2023) The selective RyR2 inhibitor ent-verticilide suppresses atrial fibrillation susceptibility caused by Pitx2 deficiency. J Mol Cell Cardiol 180: 1-9.
Knollmann B C and Roden D M (2008) A genetic framework for improving arrhythmia therapy. Nature 451(7181): 929-936.
Kober L, Torp-Pedersen C, McMurray J J, Gotzsche O, Levy S, Crijns H, Amlie J, Carlsen J and Dronedarone Study G (2008) Increased mortality after dronedarone therapy for severe heart failure. N Engl J Med 358(25): 2678-2687.
Kryshtal D O, Blackwell D J, Egly C L, Smith A N, Batiste S M, Johnston J N, Laver D R and Knollmann B C (2021) RYR2 Channel Inhibition Is the Principal Mechanism of Flecainide Action in CPVT. Circ Res 128(3): 321-331.
Laver D R, Roden L D, Ahern G P, Eager K R, Junankar P R and Dulhunty A F (1995) Cytoplasmic Ca2+ inhibits the ryanodine receptor from cardiac muscle. J Membr Biol 147(1): 7-22.
Leenhardt A, Lucet V, Denjoy I, Grau F, Ngoc D D and Coumel P (1995) Catecholaminergic polymorphic ventricular tachycardia in children. A 7-year follow-up of 21 patients. Circulation 91(5): 1512-1519.
Lipinski C A (2004) Lead- and drug-like compounds: the rule-of-five revolution. Drug Discov Today Technol 1(4): 337-341.
Marx S O, Reiken S, Hisamatsu Y, Jayaraman T, Burkhoff D, Rosemblit N and Marks A R (2000) PKA phosphorylation dissociates FKBP12.6 from the calcium release channel (ryanodine receptor): defective regulation in failing hearts. Cell 101(4): 365-376.
O'Neill E R, Sakowska M M and Laver D R (2003) Regulation of the calcium release channel from skeletal muscle by suramin and the disulfonated stilbene derivatives DIDS, DBDS, and DNDS. Biophys J 84(3): 1674-1689.
Ohshiro T, Matsuda D, Kazuhiro T, Uchida R, Nonaka K, Masuma R and Tomoda H (2012) New verticilides, inhibitors of acyl-CoA:cholesterol acyltransferase, produced by Verticillium sp. FKI-2679. J Antibiot (Tokyo) 65(5): 255-262.
Respress J L, van Oort R J, Li N, Rolim N, Dixit S S, deAlmeida A, Voigt N, Lawrence W S, Skapura D G, Skardal K, Wisloff U, Wieland T, Ai X, Pogwizd S M, Dobrev D and Wehrens X H (2012) Role of RyR2 phosphorylation at S2814 during heart failure progression. Circ Res 110(11): 1474-1483.
Seflova J, Schwarz J A, Smith A N, Svensson B, Blackwell D J, Phillips T A, Nikolaienko R, Bovo E, Rebbeck R T, Zima A V, Thomas D D, Van Petegem F, Knollmann B C, Johnston J N, Robia S L and Cornea R L (2023) RyR2 Binding of an Antiarrhythmic Cyclic Depsipeptide Mapped Using Confocal Fluorescence Lifetime Detection of FRET. ACS Chem Biol 18(10): 2290-2299.
Shiomi K, Matsui R, Kakei A, Yamaguchi Y, Masuma R, Hatano H, Arai N, Isozaki M, Tanaka H, Kobayashi S, Turberg A and Omura S (2010) Verticilide, a new ryanodine-binding inhibitor, produced by Verticillium sp. FKI-1033. J Antibiot (Tokyo) 63(2): 77-82.
Smith A N, Blackwell D J, Knollmann B C and Johnston J N (2021) Ring Size as an Independent Variable in Cyclooligomeric Depsipeptide Antiarrhythmic Activity. ACS Med Chem Lett 12(12): 1942-1947.
Terentyev D, Gyorke I, Belevych A E, Terentyeva R, Sridhar A, Nishijima Y, de Blanco E C, Khanna S, Sen C K, Cardounel A J, Carnes C A and Gyorke S (2008) Redox modification of ryanodine receptors contributes to sarcoplasmic reticulum Ca2+ leak in chronic heart failure. Circ Res 103(12): 1466-1472.
Veber D F, Johnson S R, Cheng H Y, Smith B R, Ward K W and Kopple K D (2002) Molecular properties that influence the oral bioavailability of drug candidates. J Med Chem 45(12): 2615-2623.
Waldo A L, Camm A J, deRuyter H, Friedman P L, MacNeil D J, Pauls J F, Pitt B, Pratt C M, Schwartz P J and Veltri E P (1996) Effect of d-sotalol on mortality in patients with left ventricular dysfunction after recent and remote myocardial infarction. The SWORD Investigators. Survival With Oral d-Sotalol. Lancet 348(9019): 7-12.
Watanabe H, Chopra N, Laver D, Hwang H S, Davies S S, Roach D E, Duff H J, Roden D M, Wilde A A and Knollmann B C (2009) Flecainide prevents catecholaminergic polymorphic ventricular tachycardia in mice and humans. Nature medicine 15(4): 380-383.
Watanabe H and Knollmann B C (2011) Mechanism underlying catecholaminergic polymorphic ventricular tachycardia and approaches to therapy. Journal of electrocardiology 44(6): 650-655.
Zheng Z J, Croft J B, Giles W H and Mensah G A (2001) Sudden cardiac death in the United States, 1989 to 1998. Circulation 104(18): 2158-2163.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

We claim:

1. A compound of the following formula: 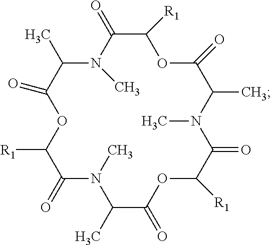
wherein each R₁ is independent and chosen from C₁-C₁₂ substituted or unsubstituted alkyl; and pharmaceutically acceptable salts thereof.

2. The compound of claim 1, wherein R₁ is C₁-C₆ alkyl.

3. The compound of claim 1, wherein R₁ is C₁-C₅ alkyl.

4. The compound of claim 1, wherein R₁ is C₅H₁₁.

5. A method of treating a subject suffering from a cardiac condition associated with RyR2, comprising administering to a subject in need thereof a RyR2 inhibiting effective amount of a compound of the following formula: 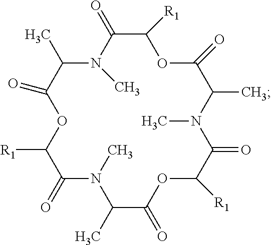
wherein each R₁ is independent and chosen from C₁-C₁₂ substituted or unsubstituted alkyl; and pharmaceutically acceptable salts thereof.

6. The method of claim 5, wherein R₁ is C₁-C₆ alkyl.

7. The method of claim 6, wherein R₁ is C₁-C₅ alkyl.

8. The method of claim 7, wherein R₁ is C₅H₁₁.

9. The method of claim 5, wherein the cardiac condition is heart failure or arrhythmia.

10. A method of treating a subject suffering from a cardiac condition associated with RyR2, comprising the step of co-administering to a subject in need thereof a RyR2 inhibiting effective amount of a compound of the following formula: 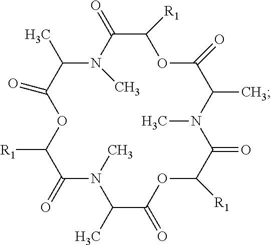
wherein each R₁ is independent and chosen from C₁-C₁₂ substituted or unsubstituted alkyl; and pharmaceutically acceptable salts thereof;
with a drug having a known effect of treating a cardiac condition.

11. The method of claim 10, wherein R₁ is C₁-C₆ alkyl.

12. The method of claim 10, wherein R₁ is C₁-C₅ alkyl.

13. The method of claim 10, wherein R₁ is C₅H₁₁.

14. The method of claim 10, wherein the cardiac condition is heart failure or arrhythmia.

15. The method of claim 10 wherein the drug is an anti-arrhythmia agent.

16. The method of claim 15, wherein the drug is a beta-blocker.
